(19) United States
(12) Patent Application Publication
(10) Pub. No.: US 2018/0263515 A1
(43) Pub. Date: Sep. 20, 2018

(54) INTRAVASCULAR CATHETER WITH PRESSURE SENSORS

(71) Applicant: ADVENTIST HEALTH SYSTEM/SUNBELT, INC., Altamonte Springs, FL (US)

(72) Inventor: Nirav Raval, Orlando, FL (US)

(21) Appl. No.: 15/923,406

(22) Filed: Mar. 16, 2018

Related U.S. Application Data
(60) Provisional application No. 62/472,203, filed on Mar. 16, 2017.

(51) Int. Cl. A61B 5/0215; A61B 5/00

(52) U.S. Cl. CPC A61B 5/02158; A61B 5/067; A61B 5/6852

(57) ABSTRACT

The present disclosure relates to an intravascular catheter that can measure intravascular pressure using MEMS sensors. Exemplary devices can be equipped with multiple MEMS pressure and temperature sensors to acquire pressure and temperature readings in multiple vascular locations. The intravascular catheter can communicate with a receiver and monitor to display sensor data.

This application claims priority under 35 USC § 119 to U.S. Provisional Application No. 62/472,203, filed Mar. 16, 2017, which is incorporated herein by reference in its entirety.

The present disclosure relates generally to a vascular catheter with micro electrical-mechanical system (MEMS) sensors.

Intravascular catheters can be equipped with mechanisms to measure variables such as pressure or temperature. In some cases, such catheters include one or more dedicated lumens with distal openings in fluid communication with transducers. Given the coagulating nature of blood during low flow and stagnant conditions, the small lumens used for pressure sensing exhibit a tendency to occlude during use. Catheters of this variety require periodic sensor zeroing and frequent in vivo calibration, which can be time-consuming or inconvenient. Further, pressure sensors dependent on transmission of pressure through a lumen can be affected by patient positioning and movement. Temperature sensors fitted in a catheter lumen can be useful in diagnostic techniques such as bolus thermodilution. Thermodilution can allow cardiac output measurement using the rate and magnitude of temperature change of fluid traveling past the temperature sensor through the catheter after a bolus of cold fluid is injected into the vasculature through a proximal port. Common temperature sensors embedded in catheters are thermistors, which are prone to failure.

There is a need for improved intravascular catheters that overcome some of the drawbacks of currently available devices. Accordingly, the present disclosure relates to a catheter with MEMS sensors, and a monitoring system for use in conjunction with such catheters, which provides advantages over existing devices.

### SUMMARY

The present disclosure relates generally to a vascular catheter with improved diagnostics capabilities. The catheter can include multiple micro electrical-mechanical system (MEMS) sensors that allow improved measurement of physiologic pressures at multiple locations. The MEMS sensors and associated systems allow pressure measurement and monitoring that is independent of patient positioning and movement.

In one embodiment, the present disclosure relates to an intravascular catheter comprising a catheter body having a proximal end and a distal end, and at least one lumen extending within and at least partially along the length of the catheter body. The intravascular catheter also includes at least one opening extending between the at least one lumen and the exterior of the catheter body, a connector hub at the distal end of the catheter body, and at least one access line affixed to the connector hub in communication with the at least one lumen of the catheter body. The intravascular catheter further comprises at least one temperature sensor and at least two pressure sensors.

In another embodiment, the present disclosure provides methods of measuring physiologic conditions comprising, selecting a catheter with at least two pressure sensors and at least one temperature sensor, creating an entryway into a patient's cardiovascular system, and inserting the distal end of the catheter into the entryway. The method of measuring physiologic conditions further comprises advancing a portion of the catheter into a heart of the patient until the distal end of the catheter is within a pulmonary artery of the patient, measuring the temperature of at least one location within the cardiovascular system, and measuring pressure of two or more locations within the cardiovascular system. The method can further comprise transmitting and recording the temperature and pressure measurements to a computing system.

The disclosed intravascular catheters provide a number of possible advantages. The reliability of MEMS sensors can be more reliable than other sensors. Furthermore, errors in thermistor readings can lead to misdiagnoses and longer hospital stays. Replacing catheters that use external transducers in fluid communication with catheter lumens reduces the risk of blood coagulation and occlusion within the lumen, and the resultant risk of inaccurate pressure readings. Bypassing the risk of lumen occlusion enables the presently described catheters to be used in patients who can benefit from longer catheter dwell times.

### BRIEF DESCRIPTION OF THE DRAWINGS

Reference will now be made to exemplary embodiments, examples of which are illustrated in the accompanying drawings. Wherever possible, the same reference numbers will be used throughout the drawings to refer to the same or like parts. The drawings are not necessarily to scale.

FIG. 1 illustrates a vascular monitoring system, according to various embodiments

FIG. 2 illustrates one embodiment of an intravascular catheter that may form part of the monitoring system shown in FIG. 1.

FIG. 3 illustrates one embodiment of various additional components of the monitoring system for receiving and displaying measurements.

FIG. 4 illustrates one embodiment of a catheter cross-section provided in accordance with the present disclosure.

FIG. 5 illustrates a catheter with MEMS sensors positioned in multiple locations, according to various embodiments of the present disclosure.

### DETAILED DESCRIPTION OF EXEMPLARY EMBODIMENTS

Reference will now be made in detail to various embodiments of the disclosed devices and methods, examples of which are illustrated in the accompanying drawings. Wherever possible, the same reference numbers will be used throughout the drawings to refer to the same or like parts.

In this application, the use of the singular includes the plural unless specifically stated otherwise. In this application, the use of “or” means “and/or” unless stated otherwise. Furthermore, the use of the term “including” as well as other forms, such as “includes” and “included,” is not limiting. Any range described herein will be understood to include the endpoints and all values between the endpoints.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

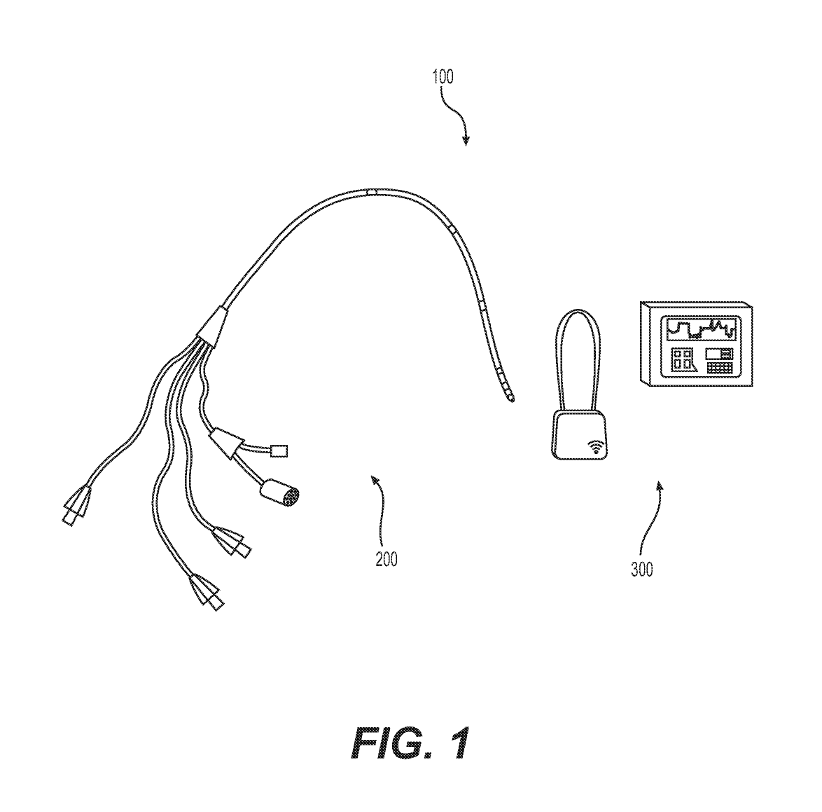

Embodiments of the present disclosure relate to a physiological monitoring system, comprising an intravascular catheter used to acquire various physiologic measurements related to vascular health, and a system to receive, record, transmit, and/or display pertinent data. The intravascular catheter is equipped with multiple sensors, including, but not limited to, MEMS pressure and temperature sensors. One embodiment of an exemplary vascular monitoring system 100 is shown in FIG. 1. As shown, the system 100 can include an intravascular catheter 200 and receiving and display means 300. The various components of the vascular monitoring system are described in more detail below.

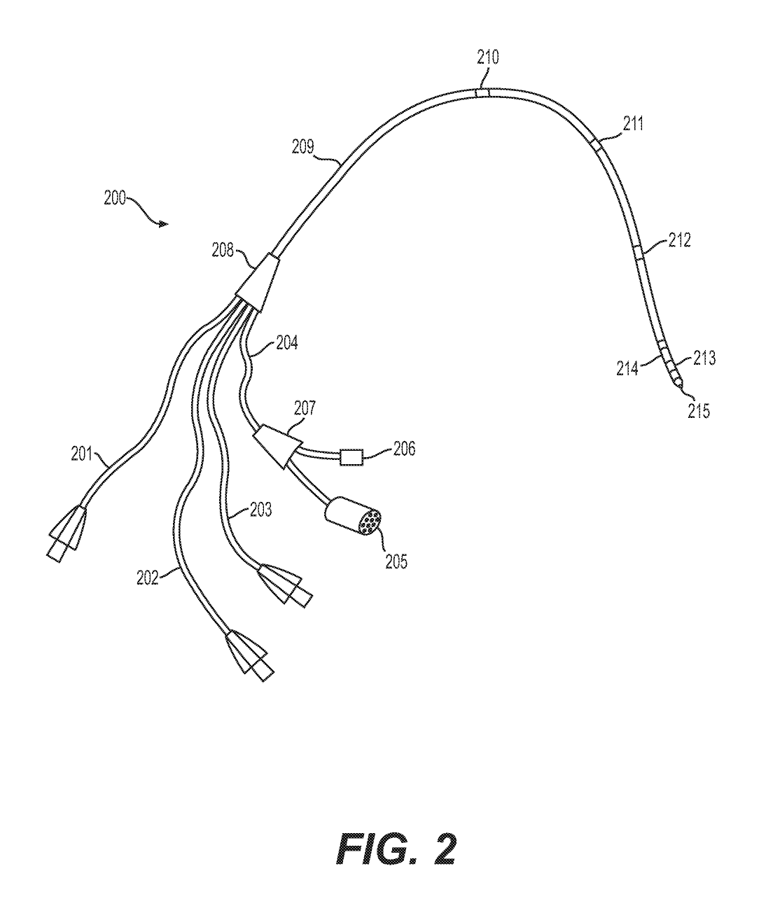

FIG. 2 illustrates an exemplary intravascular catheter 200. In some embodiments, intravascular catheter 200 comprises catheter body 209 having a proximal end and a distal end. In various embodiments, the distal end of catheter body 209 includes preformed tips, which can be provided in a variety of configurations, including, but not limited to, C-shape, S-shape, J-shape, Swan-shape or Bern-shape tips. Preformed tips can assist in accessing desired anatomic sites. According to various embodiments, catheter body 209 comprises a flexible material. In some embodiments, catheter body 209 can comprise a biocompatible polymer, elastomer, silicon, nylon, combinations of desirable materials, or any suitable biocompatible material. In various embodiments, catheter body 209 comprises at least one of polyurethane, polyethylene, polyvinylchloride, polytetrafluoroethylene, or nylon. The materials can be selected to produce desired mechanical, biologic, and/or chemical properties. For example, the materials can be selected to allow a desired stiffness/flexibility, to prevent undesired chemical reaction with physiologic fluids, or to resist or prevent infection, thrombus formation, or other adverse clinical consequences.

In some embodiments, the surfaces of catheter body 209 can be coated with a hydrophilic coating to reduce friction between catheter body 209 and various organs and tissues while the catheter is manipulated within the patient. In some embodiments, catheter body 209 can comprise a heparin-based or other anti-thrombotic coating to prevent blood clotting in and around the device during use.

In various embodiments, catheter body 209 is provided in a variety of sizes and configurations to aptly suit a variety of patient sizes and anatomies. For example, the length of catheter body 209 can measure about 50, 55, 60, 65, 70, 75, 80, 85, 90, 95, 100, 105, 110, 115, 120, 125, 130, 135, 140, 145, or 150 cm. These values may be used to define discreet catheter body 209 lengths, such as 110 cm, or ranges of lengths, such as 105-115 cm.

Additionally, catheter body 209 can be provided in a variety of diameters, defined in medicine using the French (Fr) scale, which provides catheter diameter in values equaling three times the diameter, in millimeters, thereof. In some embodiments, catheter body 209 is provided in 3, 3.5, 4, 4.5, 5, 5.5, 6, 6.5, 7, 7.5, 8, 8.5, 9, 9.5, 10, 10.5, 11, 11.5, or 12 Fr. These values may be used to define discrete catheter body 209 diameters, such as 7.5 Fr, or ranges of diameters, such as 6-7 Fr. The diameter of catheter body 209 will allow for various quantities and sizes of lumens to be extruded therein.

According to various embodiments, the intravascular catheter 200 further comprises connector hub 208 at the distal end of catheter body 209. In some embodiments, connector hub 208 comprises at least one of a y-connector or a manifold connector. Connector hub 208 connects lumen within catheter body 209 to access lines affixed to connector hub 208. In various embodiments, access lines of intravascular catheter 200 allow users to perform various functions through the catheter body 209 from outside of the body.

In various embodiments, intravascular catheter 200 may comprise multiple access lines. For example, intravascular catheter 200 may comprise two, three, four, five, six, seven or eight access lines. In some embodiments, access lines may be distinctly marked or colored to enable users to easily distinguish one access line from another. For example, access lines can be color coded. FIG. 2 illustrates four distinct access lines, 201, 202, 203, 204 in communication with lumen of catheter body 209. In some embodiments, access lines 201, 202, 203, 204 of the disclosed device may comprise single-lumen or multi-lumen tubing. In various embodiments, access lines 201, 202, 203, 204 can bifurcate, as access line 204 bifurcates into two additional access lines, 205 and 206, at a connection 207.

In some embodiments, access lines 201, 202, 203, 204 are affixed with connectors at the proximal ends thereof, such that additional devices may be attached to the access line. In some embodiments, the disclosed connectors comprise at least one of a mechanical connector, luer connector, barb connector, electronic connector, usb-type connector, or pin connector. For example, access line 201 can comprise a luer connector at its proximal end to enable attachment of a syringe thereto. In further example, access line 205 can comprise a pin connector at its proximal end to serve as a connecting means to sensors within catheter body 209.

In various embodiments, intravascular catheter 200 comprises at least one lumen extending within and at least partially along the length of catheter body 209. In various embodiments, intravascular catheter 200 comprises lumen provided in various sizes, shapes and lengths. For example, in some embodiments, intravascular catheter 200 comprises 2, 3, 4, 5, 6, 7, 8, 9, or 10 lumen. Additionally, in some embodiments, intravascular catheter 200 comprises lumens with various diameters, including about 0.05, 0.1, 0.2, 0.3, 0.4, 0.5, 0.6, 0.7, 0.8, 0.9, 1.0, 1.1, 1.2, 1.3, 1.4, or 1.5 mm in diameter.

Catheter lumens can enable multiple functions, including, but not limited to providing vascular access, drug delivery, sensor containment, access for additional catheters, and physiologic monitoring. To provide fluid communication or access to the vasculature of a patient intravascular catheter 200 comprises at least one opening extending between the at least one lumen and the exterior of catheter body 209. In some embodiments, each lumen of catheter body 209 comprises at least one opening enabling communication between the lumen and exterior of catheter body 209.

In some embodiments, catheter 209 can contain openings of various sizes in various locations along the length of the catheter body. Openings can be voids in catheter body 209 through which fluid in the lumen can flow into or out of the catheter. In some embodiments, at least one opening can serve as an internal port for the injecting medicine, administered from one of the externally located access lines 201, 202, 203, or 204.

In some embodiments, another function of intravascular catheter 200 is to provide a means to remotely inflate balloon 214 at the distal end of catheter body 209. In some embodiments, catheter body 209 comprises a balloon inflation lumen and at least one opening extending between the balloon inflation lumen and the exterior of catheter body 209. In some embodiments, balloon 214 is positioned over the at least one opening, with the distal and proximal ends of the balloon sealed to main catheter body 209, such that no fluid will migrate from the balloon to the surrounding anatomy during inflation.

In various embodiments, access line 206 is provided and connected with the balloon inflation lumen of catheter body 209 via connector hub 208. In operation, for example, a syringe or other fluid supply device can be attached to access line 206. Then, fluid, such as saline, can be injected from the proximal end of access line 206, through the balloon lumen, and then through the opening in catheter body 209, to inflate balloon 214. Balloon 214 can be used to occlude fluid flow around the catheter. For example, balloon 214 can be inflated to occlude blood flow in a vessel such that blood can only flow through intravascular catheter 200, which may be useful in certain surgical and diagnostic techniques.

An important function of intravascular catheter 200, is to provide physiological monitoring. In various embodiments, intravascular catheter 200 may contain 2, 3, 4, 5, 6, 7, or 8 sensors comprising at least one of a microelectromechanical sensor, a capacitive sensor, a piezoelectric sensor, or a combination therebetween. In some embodiments, the sensors will be MEMS type pressure sensors 210, 211, and 213, as shown on FIG. 2. In various embodiments, MEMS pressure sensors 210, 211, and 213 are positioned at least on an outer surface of catheter body 209, in an existing lumen, or in a distinct lumen in fluid communication with either the exterior of catheter body 209 or interior of an existing lumen.

The positioning of MEMS pressure sensors 210, 211, and 213 on intravascular catheter 200 can provide clinical advantages over single-sensor designs. For example, multiple pressure sensors on intravascular catheter 200, separated by certain distances, as disclosed herein, can be used to measure pressures simultaneously within different regions of the vascular system. For example, in certain embodiments, once intravascular catheter 200 is fully inserted, at least one pressure sensor is positioned to measure pressure in the pulmonary artery and at least one other pressure sensor is positioned to measure pressure in the right ventricle of a human heart. In this configuration, intravascular catheter 200 can measure the trans-pulmonary gradient, a critical physiologic measurement used to detect both advanced heart failure and pulmonary hypertension.

In some embodiments intravascular catheter 200 includes three pressure sensors. Upon final placement, intravascular catheter 200 is configured such that one pressure sensor lies within the right atrium, one pressure sensor lies within the right ventricle, and one pressure sensor lies within the pulmonary artery of a patient. Additionally, other sensor placement configurations and quantities are provided within the scope of the present disclosure to measure pressures at various anatomic locations throughout the body, such as, but not limited to the superior vena cava.

Additionally, in various embodiments, MEMS pressure sensors 210, 211, 213 and balloon 214 of intravascular catheter 200 can be used together to perform diagnostic procedures, such as pulmonary artery occlusive pressure (PAOP) measurements. PAOP measurements can aid in diagnosing various pulmonary and heart conditions, such as acute pulmonary edema, pulmonary hypertension, and left ventricular failure.

In some embodiments, intravascular catheter 200 comprises at least one temperature sensor, such as a MEMS temperature sensor 212. MEMS temperature sensor 212 can measure internal core temperatures to assist during the monitoring of fever and anesthesia-induced thermoregulatory complications. Additionally, in various embodiments, measurements from the MEMS temperature sensor 212 can be useful as a diagnostic tool in thermal dilution, a procedure performed to measure cardiac output. In various embodiments, MEMS temperature sensor 212 provides continuous, absolute temperature measurements, and aids in vital diagnostic procedures.

In some embodiments, the distal end of intravascular catheter 200 comprises atraumatic tip 215. Rounded, atraumatic tip 215 prevents trauma to surrounding tissues during movement of intravascular catheter 200, often caused from typical physiological activity, like pulsatile blood flow, catheter manipulation, or clinician manipulation. The innermost layer of organs of the vascular system is the tunica intima. If the continuous surface of the tunica intima is damaged, for example from contact by foreign devices like catheters, a thrombogenic region may form that can result in blood clotting and irregular blood flow patterns. Atraumatic tip 215 is rounded and smooth so that such tissue damage is avoided.

In some embodiments, intravascular catheter 200 can incorporate visualization and location-identifying components, for example, radiopaque markers. Such markers can allow clinicians to determine the location of devices, like catheters, within the body of the patient during a procedure, using fluoroscopy or other imaging. In various embodiments, radiopaque markers are placed at multiple locations along the catheter, including at the locations of MEMS sensors 210, 211, 212, and 213, as well as on either side of balloon 214, to allow precise positioning of intravascular catheter 200 within the vasculature of the patient.

In various embodiments, the present disclosure provides methods of measuring physiologic conditions comprising, creating an entryway into the cardiovascular system. The entryway can be established at any suitable site such as the internal jugular vein, subclavian vein, or femoral vein. Next, the method comprises inserting the distal end of intravascular catheter 200 into the entryway and advancing a portion of intravascular catheter 200 through the right atrium, into the right ventricle, and into the pulmonary artery of the patient. In some embodiments balloon 214 at the distal end of intravascular catheter 200 is inflated while intravascular catheter 200 is advanced through the cardiovascular system.

Next, the method can comprise measuring the temperature of at least one location within the cardiovascular system and measuring the pressure of two or more locations within the cardiovascular system. In various embodiments, at least one pressure sensor is positioned in the right ventricle and at least one pressure sensor is simultaneously positioned in the pulmonary artery. Further, the measurement can be transmitted, recorded, and displayed on a computing system. In various embodiments, intravascular catheter 200 is an indwelling catheter that continuously or continually monitors and records pressure and temperature.

In the clinical setting, the invasive nature of catheters, and sensitivity of catheter-mounted sensors, coupled with the desirability of patient mobility, makes continuous physiological monitoring difficult with existing devices. One such existing device includes an intravascular catheter with dedicated lumens comprising distal openings in fluid communication with transducers attached at the proximal end of the intravascular catheter. Because blood exhibits a tendency to coagulate during low flow and stagnant conditions, the small lumens dedicated for pressure sensing exhibit a tendency to occlude during use. Catheters of this type require periodic sensor zeroing and calibration, which can be time-consuming tasks. Common temperature sensors embedded in catheters are thermistors, (sensors) that are prone to failure.

For patients with precarious vascular conditions, accurate continuous or continual monitoring is exceedingly beneficial for detecting irregularities and anticipating adverse events. Thus, a catheter with multiple MEMS sensors provides an improvement over existing devices and can detect vascular pressures in numerous locations within the body, including the pulmonary artery, right ventricle, and right atrium. The accurate, real-time monitoring provided by MEMS sensors can provide rapid information on hemodynamic status.

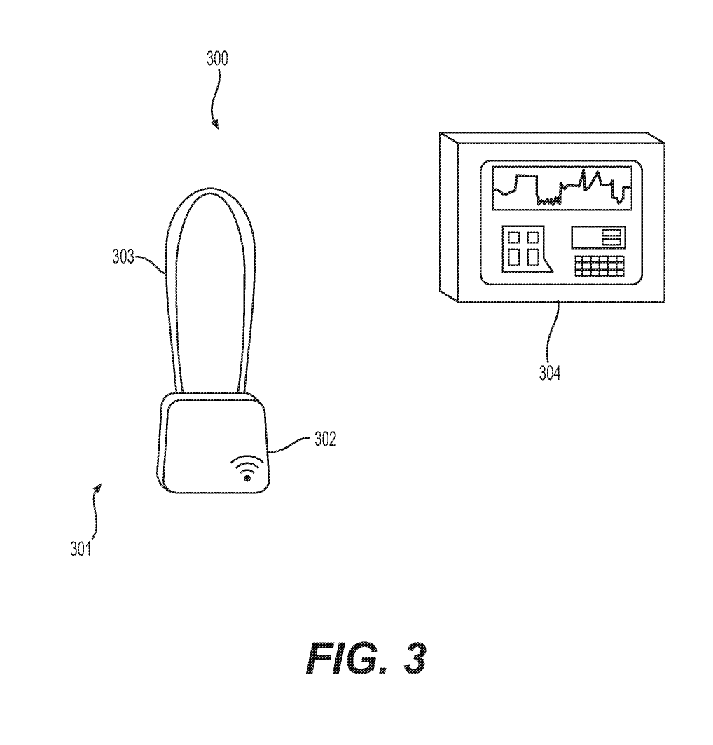

FIG. 3 illustrates an exemplary monitoring system 300. In various embodiments, exemplary monitoring system 300 may be used with intravascular catheter 200 to conduct continuous or continual physiologic monitoring. Monitoring system 300 may include a receiving means 301, and a display means 304. Receiving means 301 includes a receiver 302 and means for fixation 303 (i.e., to secure receiver 302 to a patient or stable structure). Receiver 302 can be configured to collect data from MEMS sensors 210, 211, 212, 213 in intravascular catheter 200. In one embodiment, MEMS sensors 210, 211, 212, 213 communicate with receiver 302 by a wired method. In the wired configurations, MEMS sensor 210, 211, 212, 213 leads extend from catheter body 209 to, for example, access port 205, which can then connect to receiver 302. In another embodiment, MEMS sensors 210, 211, 212, 213 in communicate with receiver 302 wirelessly by some radiofrequency. In another embodiment, MEMS sensors 210, 211, 212, 213 communicate with receiver 302 by means of a Bluetooth radio frequency band.

In one embodiment, receiving means 301 can include component 303 that provides a method for health care providers to secure receiver 302 onto, or nearby the patient. In one embodiment receiver 302 is connected to body 303 such that it can be positioned around the neck of the patient, enabling the receiver to lie in proximity to MEMS sensors 210, 211, 212, 213 of intravascular catheter 200 during a procedure. In another embodiment, receiver 302 is secured to a nearby article. In another embodiment, receiver 302 is connected to an attachment device, including, but not limited to a hip clip, arm band, or bracelet.

As noted previously, use of MEMS sensors with the present systems can allow measurement or monitoring that is independent of patient position or movement. For example, with prior art systems that require transmission of a pressure through a lumen to a sensor, the level of the sensor with respect to the measured anatomic site can have a large influence on pressure measurements. With the present system, however, the level of the receiver 302 with respect to the MEMS sensor does not adversely affect pressure readings, and therefore, provides flexibility in terms of patient positioning and mobility.

Unit 304 can be configured to display information from receiver 302. In some embodiments, unit 304 can be configured for remote monitoring of sensors in the device. Unit 304 can be monitored by health care staff such as nurses or surgeons. Unit 304 can be positioned in various areas of a health care facility, such as an operating room, nurses' station, or specialized care unit, like an intensive care unit. Unit 304 can be configured to record data for later review. Unit 304 can be provided in a variety of configurations. For example, unit 304 can be a portable, stand-alone display unit, with its own powering system, such as rechargeable batteries. Additionally, in some embodiments, unit 304 can be integratable with existing patient monitoring and imagining systems by wired or wireless means such as USB cables, or Bluetooth, and powered by a wired connection to an electrical outlet.

In a wireless communication configuration, the device can be used in a variety of diagnostic settings, which may provide an advantage over existing catheters. Previous diagnostic catheters require patients to remain in a supine position, or to remain still. Wireless signal transmission from intravascular catheter 200 to system 300 makes physiologic monitoring possible during dynamic patient states. The device of the present disclosure enables continuous physiologic monitoring while the patient is moving, either from daily human activity, or during evaluations which require movement, for example, an exercise stress test.

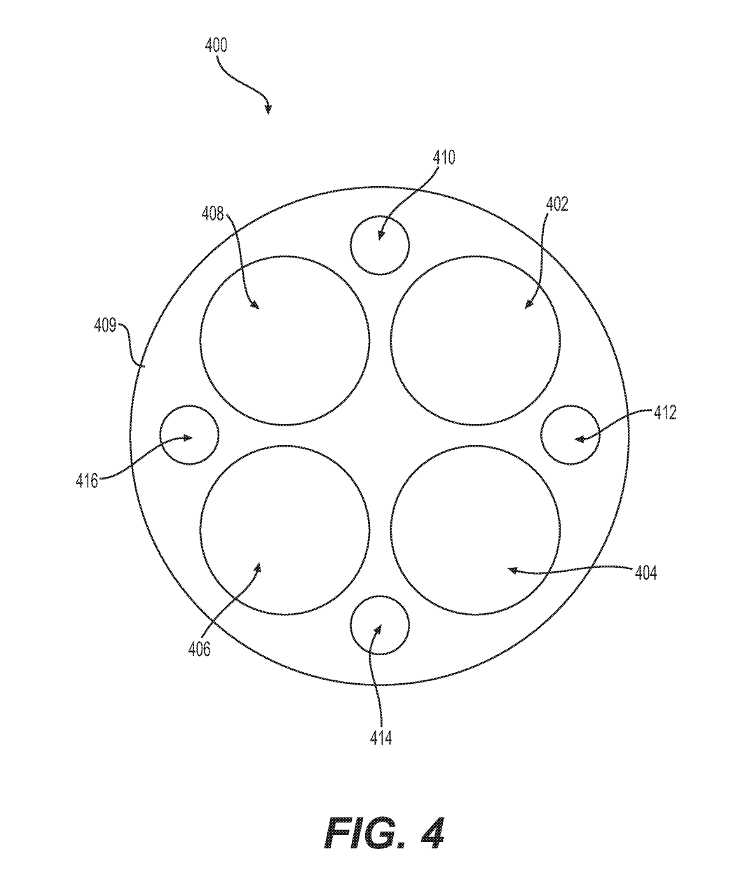

FIG. 4 illustrates a cross-sectional view of exemplary intravascular catheter 400 according to one embodiment of the present disclosure. In some embodiments, catheter body 209 is cylindrical and comprises four fluid lumens and four sensor lumens. In one embodiment, the four fluid lumens are approximately the same size, positioned with radial symmetry about the center of the cross-section of catheter body 409, and include pulmonary artery (PA) lumen 402, right ventricle (RV) lumen 404, proximal injectate lumen 406, and balloon inflation lumen 408. However, the four fluid lumen can vary in size and shape while still performing the tasks necessary of intravascular catheter 400.

It should be understood that the specific lumen configuration is exemplary and may be varied based on specific clinical goals. Accordingly, the positions and sizes of the various lumens may be modified.

Each fluid lumen is connected to an opening in catheter body 409 leading to the exterior of catheter body 409. PA lumen 402 includes an opening at the distal tip of catheter body 409, configured to lie within the pulmonary artery when intravascular catheter 400 is fully advanced. RV lumen 404 includes an opening approximately 19 cm from the distal tip of catheter body 409, configured to lie within the right ventricle when intravascular catheter 400 is fully advanced. Proximal injectate lumen 406 includes an opening approximately 30 cm from the distal tip of catheter body 409, configured to lie within the right atrium when intravascular catheter 400 is fully advanced.

It will be understood that these distances are approximate and intravascular catheter 400 can be manufactured in a variety of sizes to suit a variety of patient sizes. Thus, the distance of openings from the distal tip of catheter body 409 can vary by 10%, 25%, 50% or 100% depending on the size of the catheter, just as the anatomical distances between these anatomic locations can vary between small children and large adults.

In some embodiments, each fluid lumen within catheter body 409 will be connected, via a connector hub, to access lines on the exterior of a patient's body. These access lines are in fluid communication with the fluid lumens and provide a means for practitioners to attach external devices, such as syringes or, in the unlikely event of MEMS failure, pressure transducers, to the fluid lumens.

In various embodiments, MEMS pressure and temperature sensors can be incorporated into catheter body 409 to measure internal physiologic pressures and temperatures. MEMS sensors with wire leads require conduits to provide a passage for the leads from catheter body 409 to some electronic device, such as receiver 302 illustrated in FIG. 3. In some embodiments, catheter body 209 comprises four sensor lumens that are approximately the same size, positioned with radial symmetry about the center of the cross-section of catheter body 409, and include temperature sensor wiring lumen 410, pulmonary artery (PA) MEMS wiring lumen 412, right ventricle (RV) MEMS wiring lumen 414, and right atrium (RA) MEMS wiring lumen 416. The four sensor lumen can vary in size and shape while still performing the tasks necessary of intravascular catheter 400. The sensors themselves can be positioned in various locations on a catheter body, relative their respective wiring lumen, described in further detail below.

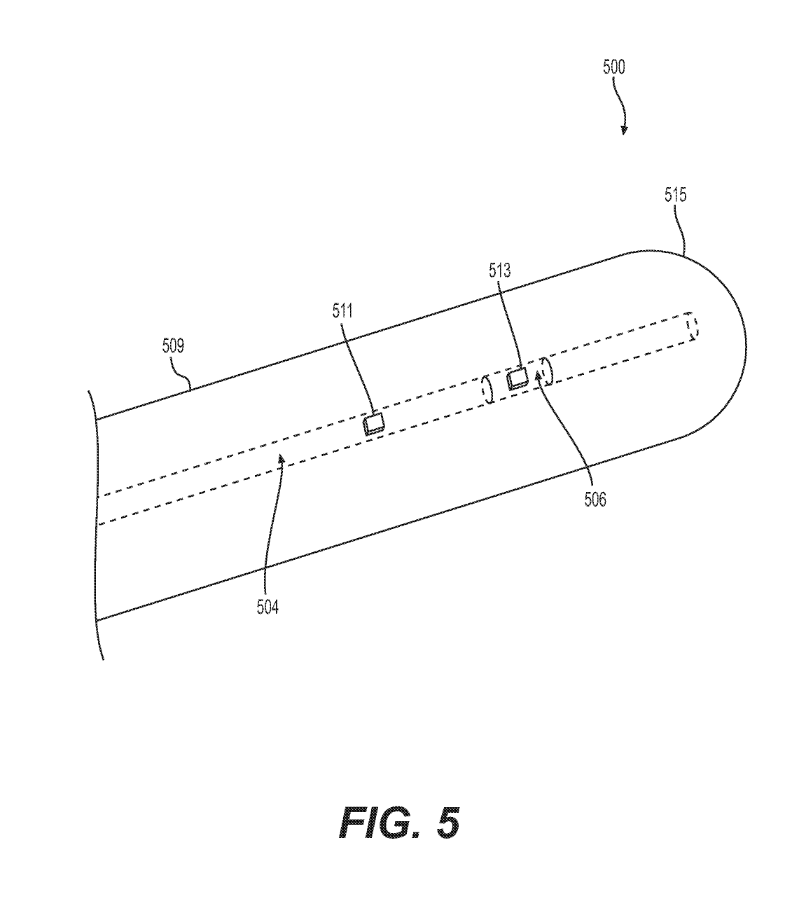

FIG. 5 illustrates MEMS sensor position options, according to various embodiments of the present disclosure. As illustrated in FIG. 5, intravascular catheter 500 comprises catheter body 509, atraumatic tip 515, wiring lumen 504, opening 506, and MEMS sensors 511 and 513. In some embodiments, MEMS sensor 511 can be mounted proximate wiring lumen 504 on the exterior surface of catheter body 509. In various embodiments, a small hole can be produced below MEMS sensor 511 leading into wiring lumen 504 so that the leads of MEMS sensor 511 can be threaded through wiring lumen 504 to an access lumen on the proximal end of intravascular catheter 500.

In some embodiments, MEMS sensor 511 can be coated with an electrically insulating material and covered with an electrically insulating material after attachment to intravascular catheter 500, e.g., to comply with federal safety regulations. Additionally, in some embodiments, a substance, like a bead of polymer can be placed around MEMS sensor 511 so that it results in a more aerodynamic surface of catheter body 509.

In some embodiments, opening 506 can be manufactured in catheter body 509 to expose wiring lumen 504 to the exterior of catheter body 509. MEMS sensor 513 can be places in wiring lumen 504 proximate opening 506. In some embodiments, the leads of MEMS sensor 513 can be threaded through wiring lumen 504. and then wiring lumen 504 can be sealed form both sides to prevent liquid from entering the lumen. As before, MEMS sensor 513 can be coated with electrically insulating materials to comply with safety standards. In some embodiments, MEMS sensor 513 can be potted in wiring lumen 504 on four sides, leaving the top side exposed to the cardiovascular system. In this embodiment, the surface of MEMS sensor 513 can be aligned with the exterior surface of catheter body 509 so that no significant disruptions in the exterior surface catheter body 509 surface are present.

In some embodiments, multiple positions and configurations of MEMS sensors throughout intravascular catheter 500 are provided to measure multiple regions of the cardiovascular system. In some embodiments, multiple sensors can share a wiring lumen.

Generally, the intravascular catheter of the present disclosure provides significant benefits over traditional intravascular catheters due to the use of more accurate and more durable MEMS pressure and/or temperature sensors. Additional embodiments and configurations of the present disclosure will be obvious to a person of ordinary skill in the art.

What is claimed is:

1. An intravascular catheter comprising:
a catheter body having a proximal end and a distal end;
at least one lumen extending within and at least partially along the length of the catheter body;
at least one opening extending between the at least one lumen and the exterior of the catheter body;
a connector hub at the distal end of the catheter body;
at least one access line affixed to the connector hub in communication with the at least one lumen of the catheter body;
at least one temperature sensor; and
at least two pressure sensors.

2. The intravascular catheter of claim 1, wherein the catheter body comprises a flexible material.

3. The intravascular catheter of claim 2, wherein the catheter body comprises at least one of a polymer, elastomer, silicon, nylon, or a combination therebetween.

4. The intravascular catheter of claim 1, wherein the catheter body comprises a balloon inflation lumen and at least one opening extending between the balloon inflation lumen and the exterior of the catheter body.

5. The intravascular catheter of claim 1, further comprising a balloon proximate the distal end of the catheter body positioned over the at least one opening extending between the balloon inflation lumen and the exterior of the catheter body, with the distal and proximal ends of the balloon are sealed to the main catheter body.

6. The intravascular catheter of claim 1, further comprising:
at least four additional lumen extending within and at least partially along the length of the catheter body; and
at least four additional openings extending between each lumen and the exterior of the catheter body.

7. The intravascular catheter of claim 1, wherein the two or more pressure sensors comprise at least one of a microelectromechanical sensor, a capacitive sensor, a piezoelectric sensor, or a combination therebetween.

8. The intravascular catheter of claim 1, wherein the two or more pressure sensors are positioned at least on an outer surface of the catheter body, in an existing lumen, or in a distinct lumen in fluid communication with either the exterior of the catheter body or interior of an existing lumen.

9. The intravascular catheter of claim 1, wherein at least one pressure sensor is positioned to measure pressure in the pulmonary artery and at least one other pressure sensor is positioned to measure pressure in the right ventricle of a human heart.

10. The intravascular catheter of claim 1, wherein the connector hub comprises at least one of a y-connector or a manifold connector.

11. The intravascular catheter of claim 1, wherein the at least one access line comprises ports affixed with connectors at the proximal ends thereof such that additional devices may be attached to the access line.

12. The intravascular catheter of claim 11, wherein the connectors at the proximal ends of the access lines comprise at least one of a mechanical connector, luer connector, barb connector, electronic connector, usb-type connector, or pin connector.

13. The intravascular catheter of claim 1, further comprising a pendant connected to the at least two pressure sensors and at least one temperature sensor to collect and process signals generated by said sensors and transmit said signals to a computing device for further processing and display, wherein the pressure measurements made by the pressure sensors are independent of patient position or movement.

14. A method of measuring physiologic conditions comprising:
selecting a catheter with at least two pressure sensors and at least one temperature sensor;
creating an entryway into a patient's cardiovascular system, inserting the distal end of the catheter into the entryway;
advancing a portion of the catheter into a heart of the patient until the distal end of the catheter is within a pulmonary artery;
measuring the temperature of at least one location within the cardiovascular system;
measuring pressure of two or more locations within the cardiovascular system; and
transmitting and recording the temperature and pressure measurements to a computing system.

15. The method of claim 14, wherein the entryway into the cardiovascular system is located in at least one of the right internal jugular vein, left subclavian vein, or femoral vein.

16. The method of claim 14, wherein advancing the catheter into the heart of the patient entails passing the distal tip of the catheter through the right atrium, into the right ventricle, and into the pulmonary artery of the patient.

17. The method of claim 14, wherein a balloon at the distal end of the catheter is inflated while the catheter is advanced through the cardiovascular system.

18. The method of claim 14, wherein at least one pressure sensor is positioned in the right ventricle and at least one pressure sensor is simultaneously positioned in the pulmonary artery.

19. The method of claim 14, wherein the catheter is an indwelling catheter that continuously or continually monitors and records pressure and temperature.

20. The method of claim 14, wherein after advancing the catheter into the cardiovascular system, an operator performs a thermodilution technique to measure cardiac output.
